(19) United States
(12) Patent Application Publication
(10) Pub. No.: US 2020/0297256 A1
(43) Pub. Date: Sep. 24, 2020

(54) ELECTRONIC DEVICE AND METHOD FOR NOTIFICATION OF BIOMETRIC INFORMATION BY ELECTRONIC DEVICE

(71) Applicant: SAMSUNG ELECTRONICS CO., LTD., Suwon-si (KR)

(72) Inventor: Hyejung SEO, Suwon-si (KR)

(21) Appl. No.: 16/811,529

(22) Filed: Mar. 6, 2020

(30) Foreign Application Priority Data
Mar. 19, 2019 (KR) 10-2019-0031241

(51) Int. Cl. A61B 5/145; A61B 5/00; G06F 3/01; G06F 9/54

(52) U.S. Cl. CPC A61B 5/14532; A61B 5/4809; A61B 5/746; G06F 9/542; A61B 5/7455; G06F 3/015; G06F 3/017; A61B 5/7405

(57) ABSTRACT

Various embodiments of the disclosure relate to an electronic device and a method for biometric information notification by the electronic device, in which the electronic device may include: a sensor module; an output device comprising output circuitry; a processor operatively connected to the sensor module and the output device; and a memory operatively connected to the processor, wherein the memory is configured to store instructions that, when executed, cause the processor to control the electronic device to: identify a blood sugar level measured at every first period via the sensor module; output a hypoglycemia prediction notification based on an amount of change in decrease of the blood sugar level exceeding a designated first value; and measure a blood sugar level at every second period, the second period being shorter than the first period, based on a blood sugar level identified after the notification is output being equal to or less than a second value.

### CROSS-REFERENCE TO RELATED APPLICATION

This application is based on and claims priority under 35 U.S.C. § 119 to Korean Patent Application No. 10-2019-0031241, filed on Mar. 19, 2019, in the Korean Intellectual Property Office, the disclosure of which is incorporated by reference herein in its entirety.

### BACKGROUND

### Field

The disclosure relates to an electronic device and a method for providing a notification associated with biometric information by the electronic device.

### Description of Related Art

Recently, various electronic devices are being developed to measure biometric information of users and to provide various health-related information on the basis of the measured biometric information. For example, measured biometric information may be used to determine whether there is an abnormality in a user's health condition.

In an electronic device, various applications (e.g., referred to as “apps”) may be installed and executed. The applications may monitor a user's health condition by periodically measuring the user's biometric information.

An electronic device may measure biometric information of a user and may provide state information related to various diseases. For example, the electronic device may determine whether a blood sugar level of the user corresponds to hypoglycemia. Hypoglycemia may occur when a patient has too much insulin, fails to eat a meal, or suddenly increases an exercise amount, and it is a side effect that occurs during diabetes treatment. When blood sugar drops, a patient has hunger, lethargy, trembles, shakes, sweats, dizziness, headaches, etc., and may lose consciousness in a severe case.

Moreover, a patient who already has hypoglycemia is more likely to relapse, and frequent occurrence of hypoglycemia may lead to decreased intelligence, a secondary accident, or the like, and may lead to loss of life in a severe case, so it is very important to predict an occurrence of hypoglycemia by monitoring pre-symptoms.

### SUMMARY

Embodiments of the disclosure provide an electronic device and a method for determining health information by the electronic device that may enable an occurrence of hypoglycemia to be predicted and may allow various notifications based on blood sugar levels to be provided, based on various context information of a user.

An electronic device according to various example embodiments of the disclosure may include: a sensor module; an output device comprising output circuitry; a processor operatively connected to the sensor module and the output device; and a memory operatively connected to the processor, wherein the memory is configured to store instructions that, when executed, cause the processor to control the electronic device to: identify a blood sugar level measured at every first period via the sensor module; output a hypoglycemia prediction notification based on an amount of change in decrease of the blood sugar level exceeding a designated first value; and measure a blood sugar level at every second period, the second period being shorter than the first period via the sensor module based on a blood sugar level identified after the notification is output being equal to or less than a second value.

A method for biometric information notification by an electronic device according to various example embodiments of the disclosure may include: identifying a blood sugar level measured at every first period; outputting a hypoglycemia prediction notification via an output device based on an amount of change in decrease of the blood sugar level exceeding a designated first value; and measuring a blood sugar level at every second period, the second period being shorter than the first period, based on a blood sugar level identified after the notification is output being equal to or less than a designated value.

According to various example embodiments of the disclosure, an electronic device and a method for biometric information notification by the electronic device may enable occurrence of hypoglycemia to be predicted, and a blood sugar measurement period and a notification output to be configured based on movement information and a biometric signal to enable the user to predict occurrence of hypoglycemia in various situations and take action against hypoglycemia.

### BRIEF DESCRIPTION OF THE DRAWINGS

The above and other aspects, features, and advantages of certain embodiments of the present disclosure will be more apparent from the following detailed description, taken in conjunction with the accompanying drawings, in which:

FIG. 1 is a block diagram illustrating an example electronic device within a network environment according to various embodiments of the disclosure;

FIG. 2 is a block diagram illustrating an example configuration of an electronic device according to various embodiments of the disclosure;

FIG. 3 is a block diagram illustrating an example electronic device according to various embodiments of the disclosure;

FIG. 4A is a diagram illustrating a front view of an example of the electronic device according to various embodiments of the disclosure;

FIG. 4B is a diagram illustrating a rear view of an example of the electronic device according to various embodiments of the disclosure;

FIG. 5 is a flowchart illustrating an example operation of providing a notification associated with biometric information by the electronic device according to various embodiments of the disclosure;

FIG. 6 is a flowchart illustrating an example operation of predicting hypoglycemia based on biometric information and providing a notification by the electronic device according to various embodiments of the disclosure;

FIG. 7 is a diagram illustrating an example biometric information prediction notification output by the electronic device according to various embodiments of the disclosure;

FIG. 8 is a flowchart illustrating an example operation of outputting a hypoglycemia prediction notification by the electronic device according to various embodiments of the disclosure;

FIG. 9 is a graph illustrating an example change in a biometric signal of a user when hypoglycemia occurs, according to various embodiments of the disclosure;

FIG. 10 is a flowchart illustrating an example operation of determining whether to output a hypoglycemia prediction notification, by the electronic device according to various embodiments of the disclosure;

FIG. 11 is a diagram illustrating an example hypoglycemia prediction notification configuration in the electronic device according to various embodiments of the disclosure;

FIG. 12 is a diagram illustrating an example hypoglycemia occurrence notification configuration in the electronic device according to various embodiments of the disclosure; and

FIG. 13 is a diagram illustrating an example data configuration for designing a hypoglycemia prediction notification model by the electronic device according to various embodiments of the disclosure.

### DETAILED DESCRIPTION

Hereinafter, various example embodiments of the disclosure will be described with reference to the accompanying drawings. The embodiments and the terms used therein are not intended to limit the technology disclosed herein to specific forms, and should be understood to include various modifications, equivalents, or alternatives to the corresponding embodiments. In describing the drawings, similar reference numerals may be used to designate similar constituent elements. A singular expression may include a plural expression unless they are definitely different in a context. In the disclosure, expressions such as “A or B” or “at least one of A or B” may include all possible combinations of items listed together. The expression “a first”, “a second”, “the first”, or “the second” may modify corresponding elements regardless of the order or the importance but does not limit the elements. When an element (e.g., first element) is referred to as being “(functionally or communicatively) connected to,” or “directly coupled” another element (second element), the element may be connected directly (e.g., via a wire) to the another element or connected to the another element via yet another element (e.g., third element).

The expression “configured to” as used in the disclosure may be interchangeably used with, for example, “suitable for”, “having the capacity to”, “designed to”, “adapted to”, “made to”, or “capable of” in terms of hardware or software, according to circumstances. In some situations, the expression “device configured to” may mean that the device, together with other devices or components, “is able to”. For example, the phrase “processor adapted (or configured) to perform A, B, and C” may mean a dedicated processor (e.g. embedded processor) only for performing the corresponding operations or a generic-purpose processor (e.g., central processing unit (CPU) or application processor (AP)) that can perform the corresponding operations by executing one or more software programs stored in a memory device.

An electronic device according to various embodiments of the disclosure may include at least one of, for example, a smart phone, a tablet Personal Computer (PC), a mobile phone, a video phone, an electronic book reader (e-book reader), a desktop PC, a laptop PC, a netbook computer, a workstation, a server, a personal digital assistant (PDA), a portable multimedia player (PMP), an MPEG-1 audio layer-3 (MP3) player, a mobile medical device, a camera, and a wearable device. According to various embodiments, the wearable device may include at least one of an accessory type (e.g., a watch, a ring, a bracelet, an anklet, a necklace, a pair of glasses, a contact lens, or a head-mounted device (HMD)), a fabric or clothing integrated type (e.g., an electronic clothing), a body-mounted type (e.g., a skin pad, or tattoo), and a bio-implantable type (e.g., an implantable circuit). In some embodiments, the electronic device may include at least one of, for example, a television, a digital video disk (DVD) player, an audio player, a refrigerator, an air conditioner, a vacuum cleaner, an oven, a microwave oven, a washing machine, an air cleaner, a set-top box, a home automation control panel, a security control panel, a TV box (e.g., Samsung HomeSync™, Apple TV™, or Google TV™), a game console (e.g., Xbox™ and PlayStation™), an electronic dictionary, an electronic key, a camcorder, and an electronic photo frame.

In other embodiments, the electronic device may include at least one of various medical devices (e.g., various portable medical measuring devices (a blood glucose monitoring device, a heart rate monitoring device, a blood pressure measuring device, a body temperature measuring device, etc.), a magnetic resonance angiography (MRA), a magnetic resonance imaging (MRI), a computed tomography (CT) machine, and an ultrasonic machine), a navigation device, a global navigation satellite system (GNSS), an event data recorder (EDR), a flight data recorder (FDR), a vehicle infotainment device, an electronic device for a ship (e.g., a navigation device for a ship, and a gyro-compass), avionics, security devices, an automotive head unit, a robot for home or industry, a drone, an automatic teller's machine (ATM) in banks, point of sales (POS) in a shop, or internet device of things (e.g., a light bulb, various sensors, a sprinkler device, a fire alarm, a thermostat, a streetlamp, a toaster, a sporting goods, a hot water tank, a heater, a boiler, etc.). According to some embodiments, an electronic device may include at least one of a part of furniture a building/structure, or a vehicle, an electronic board, an electronic signature receiving device, a projector, and various types of measuring instruments (e.g., a water meter, an electric meter, a gas meter, a radio wave meter, and the like). In various embodiments, the electronic device may be flexible, or may be a combination of one or more of the aforementioned various devices. The electronic device according to embodiments of the disclosure is not limited to the above-described devices. In the disclosure, the term “user” may indicate a person using an electronic device or a device (e.g., an artificial intelligence electronic device) using an electronic device.

Hereinafter, an electronic device and a method for outputting a content according to various example embodiments of the disclosure will be described in greater detail with reference to the accompanying drawings. In the disclosure, the term “user” may indicate a person using an electronic device or a device (e.g., an artificial intelligence electronic device) using an electronic device.

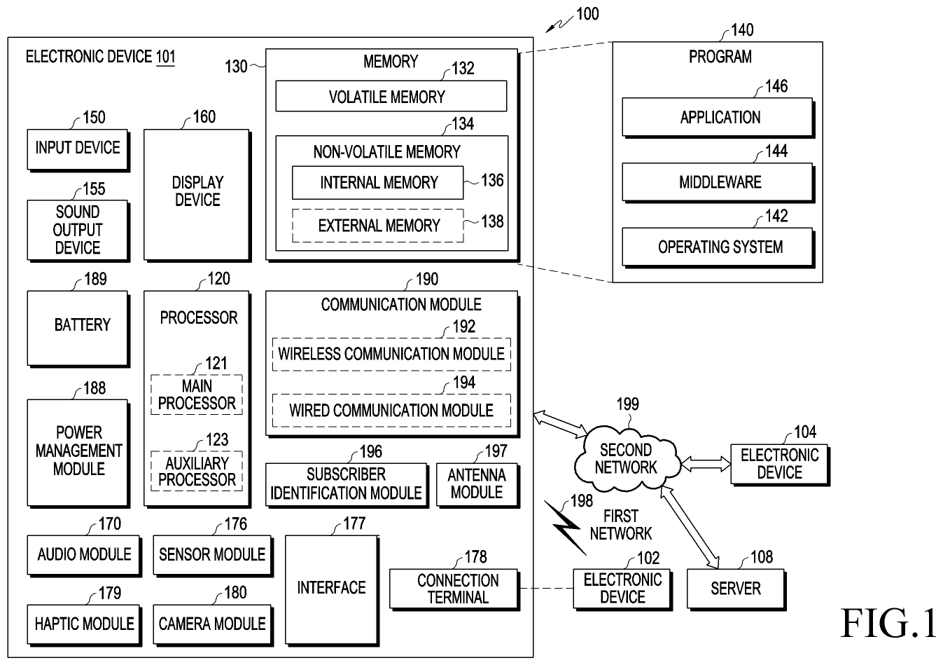

FIG. 1 is a block diagram illustrating an example electronic device 101 within a network environment 100 according to various embodiments of the disclosure.

Referring to FIG. 1, the electronic device 101 in the network environment 100 may communicate with an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input device 150, a sound output device 155, a display device 160, an audio module 170, a sensor module 176, an interface 177, a haptic module 179, a camera module 180, a power management module 188, a battery 189, a communication module 190, a subscriber identification module (SIM) 196, or an antenna module 197. In some embodiments, at least one (e.g., the display device 160 or the camera module 180) of the components may be omitted from the electronic device 101, or one or more other components may be added in the electronic device 101. In some embodiments, some of the components may be implemented as single integrated circuitry. For example, the sensor module 176 (e.g., a fingerprint sensor, an iris sensor, or an illuminance sensor) may be implemented as embedded in the display device 160 (e.g., a display).

The processor 120 may execute, for example, software (e.g., a program 140) to control at least one other component (e.g., a hardware or software component) of the electronic device 101 coupled with the processor 120, and may perform various data processing or computation. According to an example embodiment, as at least part of the data processing or computation, the processor 120 may load a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), and an auxiliary processor 123 (e.g., a graphics processing unit (GPU), an image signal processor (ISP), a sensor hub processor, or a communication processor (CP)) that is operable independently from, or in conjunction with, the main processor 121. Additionally or alternatively, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display device 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input device 150 may receive a command or data to be used by other component (e.g., the processor 120) of the electronic device 101, from the outside (e.g., a user) of the electronic device 101. The input device 150 may include, for example, a microphone, a mouse, a keyboard, or a digital pen (e.g., a stylus pen).

The sound output device 155 may output sound signals to the outside of the electronic device 101. The sound output device 155 may include, for example, a speaker or a receiver. The speaker may be used for general purposes, such as playing multimedia or playing record, and the receiver may be used for an incoming calls. According to an embodiment, the receiver may be implemented as separate from, or as part of the speaker.

The display device 160 may visually provide information to the outside (e.g., a user) of the electronic device 101. The display device 160 may include, for example, a display, a hologram device, or a projector and control circuitry to control a corresponding one of the display, hologram device, and projector. According to an embodiment, the display device 160 may include touch circuitry adapted to detect a touch, or sensor circuitry (e.g., a pressure sensor) adapted to measure the intensity of force incurred by the touch.

The audio module 170 may convert a sound into an electrical signal and vice versa. According to an embodiment, the audio module 170 may obtain the sound via the input device 150, or output the sound via the sound output device 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an example embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a cellular network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., PCB). According to an embodiment, the antenna module 197 may include a plurality of antennas. In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 and 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, or client-server computing technology may be used, for example.

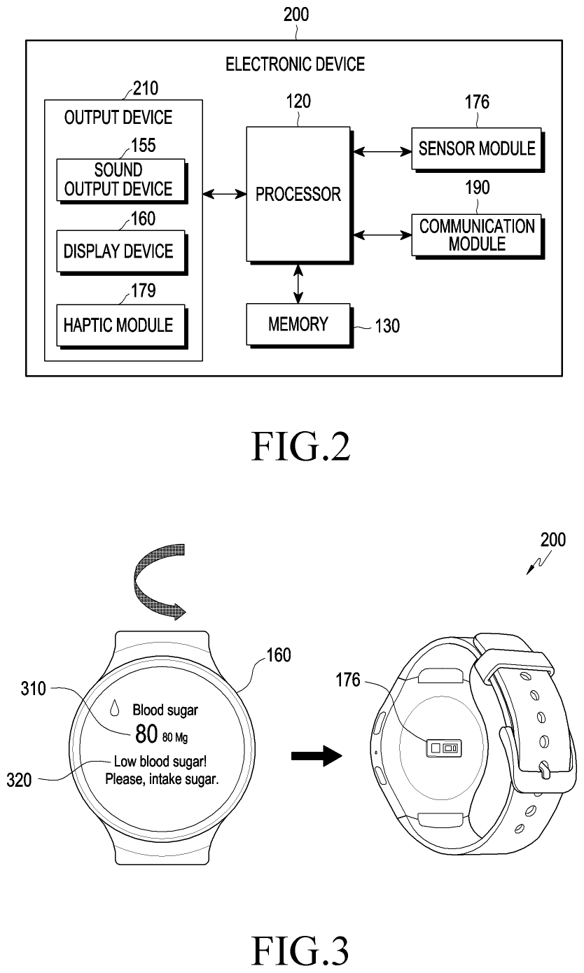

FIG. 2 is a block diagram illustrating an example configuration of an electronic device according to various embodiments of the disclosure.

Referring to FIG. 2, an electronic device 200 (e.g., the electronic device 101 or 102) may include at least one among the processor (e.g., including processing circuitry) 120, the memory 130, the sensor module 176, the communication module (e.g., including communication circuitry) 190, and an output device (e.g., including output circuitry) 210. The output device 210 may include various output circuitry, such as, for example, and without limitation, at least one among the sound output device (e.g., including sound output circuitry) 155, the display device 160, and the haptic module 179.

According to various embodiments of the disclosure, the electronic device 200 may measure a biometric signal via the sensor module 176, or may receive, via the communication module 190, biometric information measured by an external electronic device (e.g., the electronic device 102).

According to various embodiments of the disclosure, the processor 120 may include various processing circuitry and control overall operations of the electronic device 200. For example, the processor 120 may determine the possibility of hypoglycemia based on various information related to a user, and may provide a notification related to hypoglycemia to the user.

According to various embodiments of the disclosure, the processor 120 may determine a movement state of the user. For example, the processor 120 may determine whether the user is exercising, inactive (sedentary), sleeping, eating, or drinking.

According to various embodiments of the disclosure, the processor 120 may check a health record of the user. For example, the health record may be input by the user or may be transmitted from an external electronic device (e.g., a medical institution). The health record may include the user's gender, age, height, weight, disease information (e.g., the presence or absence of diabetes, or a frequency of occurrence of hypoglycemia), medication information, race, family history, the presence or absence of an allergy, or the like, and may further include various information related to the health of the user.

According to various embodiments of the disclosure, the processor 120 may check environment information of the user. The environment information may include information related to a surrounding environment of the electronic device 200, and may include GPS information, temperature information, or humidity information.

According to various embodiments of the disclosure, the display device 160 may output image or text data. For example, the processor 120 may predict biometric information of the user, and may control the display 160 to output a notification relating to the prediction in the form of image or text data.

According to various embodiments of the disclosure, the sensor module 176 may include, for example, and without limitation, at least one of a gyro sensor, an acceleration sensor, a grip sensor, a proximity sensor, a temperature sensor, or a biometric sensor, and may include various sensors that sense the user's movement or biometric information.

According to various embodiments of the disclosure, the biometric sensor may sense a biometric signal of the user when the biometric sensor non-invasively contacts (or approaches) a part of the user's body. The processor 120 may determine, based on the biometric signal, at least one of electrocardiogram (ECG), photoplethysmography (PPG), impedance plethysmography, body temperature, blood sugar, electroencephalogram (EEG), blood pressure, heart rate (HR), or sweat components.

According to various embodiments of the disclosure, the processor 120 may determine the user's movement state based on a signal sensed via the sensor module 176. For example, the processor 120 may determine a movement state in various situations, such as whether the user is exercising, inactive, sleeping, or eating, based on a signal of movement measured by the sensor module 176.

According to various embodiments of the disclosure, the processor 120 may determine an appropriate blood sugar level, a direction of change in blood sugar, or a blood sugar change rate according to the user's movement state. For example, the processor 120 may predict occurrence of hypoglycemia, based on a blood sugar level that is lower than the appropriate blood sugar level, a direction of change in blood sugar, which indicates a decrease, or a blood sugar change rate that is equal to or greater than a designated value, in a state where the user is exercising, inactivity, sleep, or eating.

According to various embodiments of the disclosure, even when the user usually shows a normal blood sugar level, glucose concentrations in the blood may be lowered due to sudden exercise, and temporary hypoglycemia may occur. For example, the processor 120 may determine whether the user has experienced hypoglycemia, based on the user's health information, and may predict whether hypoglycemia is likely to be continued after the user finishes exercise.

According to various embodiments of the disclosure, if the possibility of occurrence of hypoglycemia is predicted, the processor 120 may output various types of data (e.g., image data or vibration data) to induce the user to stop exercising and measure blood sugar.

According to various embodiments of the disclosure, the user may develop nighttime hypoglycemia with an extremely low blood sugar level while sleeping. For example, the processor 120 may determine that the user is sleeping, based on a movement state, and may determine whether night hypoglycemia occurs.

According to various embodiments of the disclosure, if occurrence of night hypoglycemia is expected based on health information of the user, the processor 120 may output an alarm (e.g., vibration data) to allow the user to measure blood sugar at a specific time (e.g., 2 to 3 am), or may automatically measure blood sugar.

According to various embodiments of the disclosure, the processor 120 may configure a measurement period according to a hypoglycemia occurrence state, to measure a blood sugar level. For example, the hypoglycemia occurrence state may include a non-occurrence state, an initial hypoglycemia state, a hypoglycemia predicted state, a hypoglycemia occurrence confirmed state, or a recovering state.

According to various embodiments of the disclosure, the non-occurrence state may indicate a user state in which hypoglycemia has not previously occurred. For example, the processor 120 may perform control to measure blood sugar every first period (e.g., one week).

According to various embodiments of the disclosure, the initial hypoglycemia state may indicate a user state in which a blood sugar level is lower than a usual blood sugar level. For example, the processor 120 may perform control to measure blood sugar at every second period (e.g., five minutes) which is shorter than the first period.

According to various embodiments of the disclosure, the hypoglycemia predicted state may indicate a state in which occurrence of hypoglycemia is predicted based on previously measured blood sugar information when the rate of decrease in blood sugar level is equal to or greater than a designated value. For example, the processor 120 may output a notification informing prediction of occurrence of hypoglycemia, and may perform control to measure blood sugar at every third period (e.g., one minute) which is shorter than the second period, thereby rapidly measuring a change in blood sugar of the user.

According to various embodiments of the disclosure, the hypoglycemia occurrence confirmation state may be a state in which the user's blood sugar level is measured to be lower than a value corresponding to hypoglycemia, so that it is determined that hypoglycemia has occurred in the user. For example, the processor 120 may perform control to measure blood sugar at every fourth period (e.g., 30 seconds) which is shorter than the third period.

According to various embodiments of the disclosure, if it is determined that the user is in the hypoglycemia predicted state or the hypoglycemia occurrence confirmed state, the processor 120 may output a notification encouraging the user to intake sugar (e.g., cola, orange juice, milk, sugar cubes, etc.) which enables fastest absorption of blood sugar. For example, if a user input for the notification is not received, the processor 120 may make an emergency call or may transmit information related to the occurrence of hypoglycemia of the user to a designated contact or a medical institution.

According to various embodiments of the disclosure, the recovering state may be a state in which the user previously had hypoglycemia, but the user's blood sugar level has been measured to be normal for a predetermined period of time. For example, based on checking of a previous hypoglycemia occurrence history for the user, the processor 120 may perform control to measure blood sugar at every fifth period (e.g., 24 hours) which is longer than the second period that is shorter than the first period.

According to various embodiments of the disclosure, the electronic device 200 may determine a hypoglycemia occurrence state based on various determination conditions, may configure a blood sugar measurement period according to the hypoglycemia occurrence state, and may output a hypoglycemia prediction notification.

Table 1 relates to an output configuration of a hypoglycemia prediction notification and a blood sugar measurement period preconfigured for the hypoglycemia occurrence state according to various embodiments of the disclosure.

TABLE 1

| State | State determination | Measurement | | Notification output |
|---|---|---|---|---|
| classifications | conditions | periods | | configurations |
| Non- | Determine occurrence of | 5 | minutes | No notification |
| occurrence | movement information | | | |
| state | (e.g., treadmill | | | |
| | movement, walking, | | | |
| | running, hiking, etc.) | | | |
| | Confirm biometric | | | |
| | signals (e.g., blood sugar, | | | |
| | heart rate, amount of | | | |
| | sweat, body temperature, | | | |
| | and blood pressure) to be | | | |
| | normal | | | |
| Hypoglycemia | Blood sugar level being | 1 | minute | Recommended |
| predicted state | measured to a value | | | glucose intake (15 g of |
| | below 100 mg/dl | | | sugar) |
| | Blood sugar level being | | | Provide notifications |
| | decreased by 1 mg/dl or | | | of hypoglycemia |
| | more per minute | | | occurrence possibility |
| | Check changes in | | | and predicted |
| | biometric signals | | | occurrence time |
| | | | | information |
| Hypoglycemia | Blood sugar level being | 30 | seconds | Recommended |
| occurrence | measured to a value of | | | glucose intake (15 g of |
| state | 70 mg/dl or lower | | | sugar) |
| | Check changes in | | | Transmit biometric |
| | biometric signals | | | signal information and |
| | | | | blood sugar |
| | | | | information to |
| | | | | designated contacts and |
| | | | | medical institutions |
| Recovering | Blood sugar level being | 5 | minutes | No notification |
| state | measured to a value of 70 | | | Stop output of |
| | mg/dl or higher for a | | | hypoglycemia |
| | designated time (e.g., 30 | | | prediction notifications |
| | minutes) | | | Notify that output of |
| | | | | hypoglycemia |
| | | | | prediction notifications |
| | | | | to designated contacts |
| | | | | has stopped |

The sound output device 155 may include various sound output circuitry and output voice data. For example, the processor 120 may control the sound output device 155 to output voice data including a content of hypoglycemia prediction or a content of occurrence of hypoglycemia. According to various embodiments of the disclosure, the processor 120 may predict biometric information of the user, and may control the sound output device 155 to output voice data for notification of the prediction. For example, the processor 120 may control the voice data to be output by configuring a tone or a volume according to importance of biometric information prediction.

The haptic module 179 may output vibration data. For example, the processor 120 may control the haptic module 179 to output vibration data to notify of hypoglycemia prediction or occurrence of hypoglycemia.

According to various embodiments of the disclosure, the processor 120 may predict biometric information of the user, and may control the haptic module 179 to output vibration data for notification of the prediction. For example, the processor 120 may control the vibration data to be output by configuring a vibration period or a vibration intensity according to importance of biometric information prediction.

The communication module 190 may include various communication circuitry and perform data communication with an external electronic device. For example, if it is predicted that a blood sugar level of the user is measured to a designated value or lower within a predetermined time, the processor 120 may control the communication module 190 so that a message including the predicted information is transmitted to a designated contact. The designated contact may include a predesignated specific user's contacts or contact numbers of medical institutions.

According to various embodiments of the disclosure, after the message is transmitted, if a user input is not received within a predesignated time or if the blood sugar level is measured to the designated value or lower, the processor 120 may determine that the user is unable to take self-measurement against hypoglycemia. For example, the processor 120 may control the communication module 190 to transmit an emergency message to a contact number (e.g., 911) of a rescue agency.

The electronic device 200 according to various example embodiments of the disclosure may include: the sensor module; the output device (e.g., the display device 160); the processor operatively connected to the sensor module and the output device; and the memory operatively connected to the processor, wherein the memory is configured to store instructions that, when executed, cause the processor to control the electronic device to: check a blood sugar level of a user, which is measured at every first period via the sensor module; output a hypoglycemia prediction notification via the output device based on an amount of change in decrease of the blood sugar level exceeding a designated first value; and measure a blood sugar level at every second period, the second period being shorter than the first period, via the sensor module based on a blood sugar level checked after the notification is output being equal to or less than a second value.

The instructions according to various example embodiments of the disclosure may, when executed, cause the processor to control the electronic device to output a first notification informing the occurrence of hypoglycemia via the output device based on occurrence of hypoglycemia being determined based on the blood sugar level measured at every second period.

The instructions according to various example embodiments of the disclosure may, when executed, cause the processor to control the electronic device to output the first notification via the output device at every second period.

The instructions according to various example embodiments of the disclosure may, when executed, cause the processor to control the electronic device to output the first notification via the output device at every third period corresponding to the amount of change in decrease of the blood sugar level.

The electronic device according to various example embodiments of the disclosure may further include the communication module comprising communication circuitry, wherein the instructions may, when executed, cause the processor to control the electronic device to control the communication module to make an emergency call based on a user input for the first notification not being received within a predesignated time.

The output device according to various example embodiments of the disclosure may further include the haptic module or the sound output device, wherein a data type included in the hypoglycemia prediction notification includes at least one of vibration data output via the haptic module, voice data output via the sound output device, text output via the display device, or image data output via the display device.

The instructions according to various example embodiments of the disclosure may, when executed, cause the processor to control the electronic device to: determine, via the sensor module, that the user is in a sleeping state; and in the sleeping state in which the user is sleeping, output the voice data or vibration data, which is configured to induce the user to wake up, via the output device based on a rate of decrease in the blood sugar level measured at every first period exceeding a designated value.

The instructions according to various example embodiments of the disclosure may, when executed, cause the processor to control the electronic device to output the hypoglycemia prediction notification including a content that induces glucose intake via the output device based on determining, via the sensor module, that the user is in a wake-up state.

The instructions according to various example embodiments of the disclosure may, when executed, cause the processor to control the electronic device to output the hypoglycemia prediction notification at every configured period via the output device after the hypoglycemia prediction notification is output based on a predesignated user input not being received within a designated time.

FIG. 3 is a block diagram illustrating an example electronic device according to various embodiments of the disclosure.

Referring to FIG. 3, the electronic device 200 may, for example, be a watch-type wearable device. For example, the electronic device 200 may include the display device 160 and the sensor module 176.

For example, the sensor module 176 may be configured to be disposed on the rear surface of the electronic device 200 and configured to acquire a biometric signal by touching or approaching a body part (e.g., skin) of a user when worn on the user's wrist.

According to various embodiments of the disclosure, the electronic device 200 may output a notification related to a biometric signal via the display device 160. For example, if the biometric signal indicates a blood sugar level lower than the designated value or a change of rapidly decreasing blood sugar, the electronic device 200 may output a notification informing the possibility of occurrence of hypoglycemia.

According to various embodiments of the disclosure, the notification may be displayed in the form of an image on the display device 160. For example, the notification may include a measured blood sugar level 310 of the current user and a content 320 (e.g., “Low blood sugar! Please, intake sugar”) recommending glucose intake to increase the measured blood sugar level.

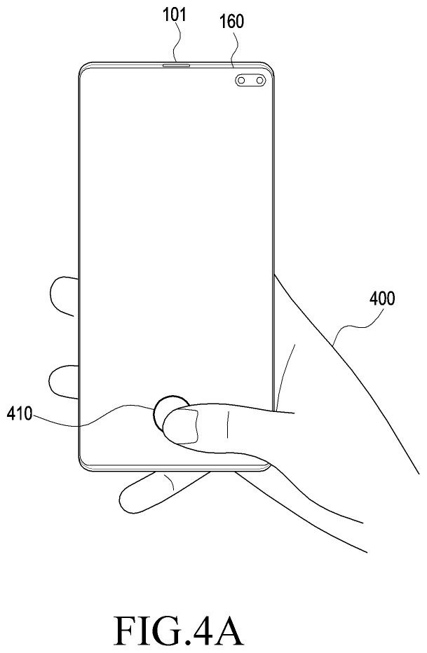

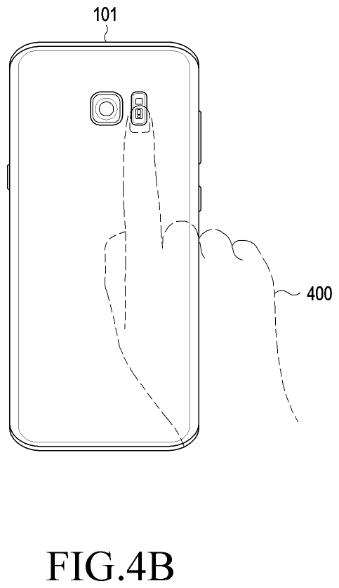

FIG. 4A is a diagram illustrating a front view of an example electronic device according to various embodiments of the disclosure.

Referring to FIG. 4A, the electronic device 101, in the form of a smartphone, may sense proximity (or touch) of a part of the body of a user 400 via the sensor module 176. For example, the sensor module 176 may be included in the display device 160 to sense a touch input approaching the display device 160.

According to various embodiments of the disclosure, the display device 160 may include a touch sensor and a touch sensor IC configured to control the touch sensor. The touch sensor IC may control the touch sensor to sense, for example, a touch input or a hovering input to a specific position of a display of the display device 160. For example, the touch sensor IC may sense a touch input or a hovering input by measuring a change in a signal (e.g., voltage, quantity of light, resistance, or quantity of charge) for a specific position of the display. The touch sensor IC may provide the processor 120 with information (e.g., position, area, pressure, or time) relating to the sensed touch input or hovering input.

According to various embodiments of the disclosure, the display device 160 may further include at least one sensor of the sensor module 176 or a control circuit therefor. The at least one sensor or the control circuit therefor may be embedded in a part of the display device 160 or a part of the touch circuit. For example, when the sensor module 176 embedded in the display device 160 includes a biometric sensor (e.g., a PPG sensor), the biometric sensor may acquire biometric information (e.g., an amount of blood sugar) associated with a touch input via a partial area of the display of the display device 160, which is exposed to the outside.

According to various embodiments of the disclosure, the at least one sensor may be disposed between pixels of a pixel layer of the display, or above or below the pixel layer. For example, the electronic device 101 may control the display device 160 to display an interface 410 configured to measure biometric information (e.g., an amount of blood sugar), wherein the interface 410 may include text for inducing contact of a part of the body, which is for measurement of biometric information, and an object indicating a part where the part of the body is in contact.

FIG. 4B is a diagram illustrating a rear view of an example electronic device according to various embodiments of the disclosure.

Referring to FIG. 4B, the electronic device 101, in the form of a smartphone, may sense proximity (or touch) of a part of the body of a user 400 via the sensor module 176. For example, the sensor module 176 may include a light emission unit and a light reception unit.

According to various embodiments of the disclosure, if proximity of the user 400 is sensed via the proximity sensor of the sensor module 176, the electronic device 101 may control the light emission unit to output (or emit), to the user, a signal (e.g., light) for acquisition of information for a blood sugar level in blood. For example, transmitted light or light reflected by the output light may be received in the light reception unit.

According to various embodiments of the disclosure, the electronic device 200 may measure the amount of light received in the light reception unit to measure a biometric signal of the user. For example, the electronic device 200 may measure a blood pressure or a heart rate by performing photoplethysmograph measurement (or photoplethysmography).

According to various embodiments of the disclosure, the electronic device 200 may output a notification informing the user to stop exercising or encouraging the user to ingest food that can immediately raise blood sugar, based on measured biometric information and whether the user has hypoglycemia. For example, if no user input is identified within a designated time after the output of the notification, the electronic device 200 may control an emergency message to be transmitted to a designated contact.

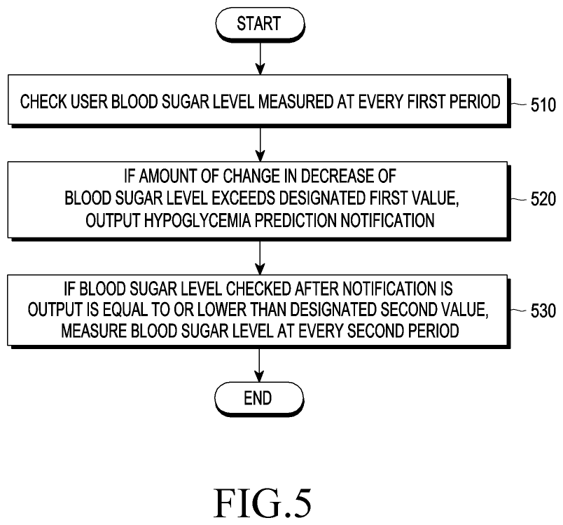

FIG. 5 is an flowchart illustrating an example operation of providing a notification associated with biometric information by an electronic device according to various embodiments of the disclosure.

Referring to FIG. 5, in operation 510, an electronic device (e.g., the electronic device 101 of FIG. 1, FIG. 4A, or FIG. 4B or the electronic device 200 of FIG. 2 and FIG. 3) may check a blood sugar level of a user, which may be measured at every first period.

In operation 520, if the amount of change in decrease of the blood sugar level exceeds a designated first value, the electronic device may output a hypoglycemia prediction notification.

In operation 530, if the blood sugar level checked after the output of the notification is equal to or lower than a designated second value, the electronic device may measure a blood sugar level at every second period, the second period being less than the first period.

A method for providing a biometric information notification by the electronic device according to various example embodiments of the disclosure may further include: outputting a first notification informing the occurrence of hypoglycemia based on occurrence of hypoglycemia being determined based on the blood sugar level measured at every second period; and outputting the first notification at every second period.

The method for providing a biometric information notification by the electronic device according to various example embodiments of the disclosure may further include outputting the first notification at every third period corresponding to the amount of change in decrease of the blood sugar level.

The method for providing a biometric information notification by the electronic device according to various example embodiments of the disclosure may further include making an emergency call if no user input is received in response to the first notification within a predesignated time.

A data type included in the hypoglycemia prediction notification according to various example embodiments of the disclosure may include at least one of vibration data output via the haptic module 179, voice data output via the sound output device 155, text output via the display device 160, or image data output via the display device 160.

The method for providing a biometric information notification by the electronic device according to various example embodiments of the disclosure may further include: determining that the user is in a sleeping state; and in the sleeping state in which the user is sleeping, outputting the voice data or vibration data, which induces the user to wake up based on a rate of decrease in the blood sugar level measured at every first period exceeding a designated value.

The method for providing a biometric information notification by the electronic device according to various example embodiments of the disclosure may further include, outputting the hypoglycemia prediction notification including a content that induces glucose intake based on it being determined that the user is in a wake-up state.

The method for providing a biometric information notification by the electronic device according to various example embodiments of the disclosure may further include, outputting the hypoglycemia prediction notification at every configured period based on no predesignated user input being received within a designated time after the output of the hypoglycemia prediction notification.

The method for providing a biometric information notification by the electronic device according to various example embodiments of the disclosure may further include: measuring a biometric signal for the user; and determining whether hypoglycemia has occurred in the user based on a pattern of the measured biometric signal.

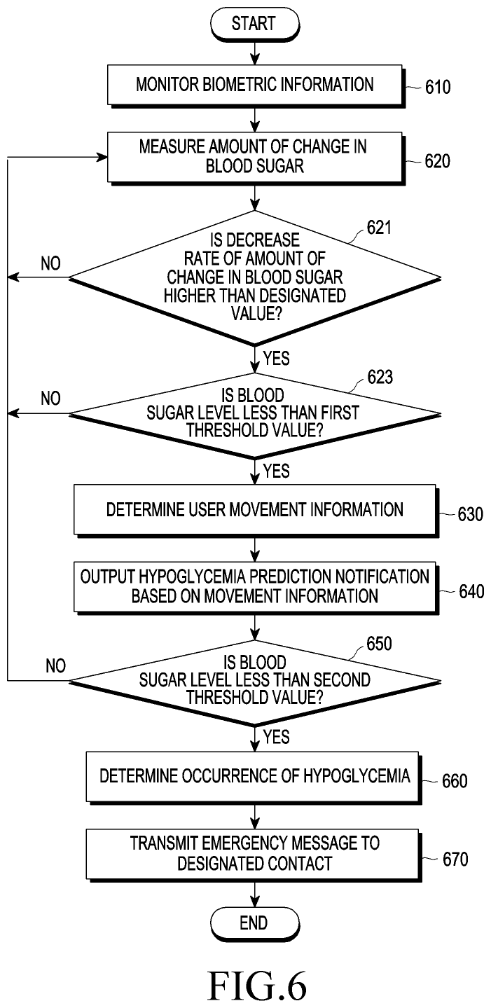

FIG. 6 is a flowchart illustrating an example operation of predicting hypoglycemia based on biometric information and providing a notification by the electronic device according to various embodiments of the disclosure.

Referring to FIG. 6, in operation 610, the electronic device 200 may monitor biometric information of a user. The biometric information may include information indicating the user's blood sugar level, heart rate, blood pressure, stress (heart rate variability), or sweat amount.

In operation 620, the electronic device 200 may measure the amount of change in blood sugar of the user. For example, the electronic device 200 may measure the amount of change in blood sugar at every designated period (e.g., at every 5 minutes).

In operation 621, the electronic device 200 may determine whether the rate of blood sugar decrease exceeds a designated value (e.g., 1 mg/dl/min).

In operation 621, if the amount of change in blood sugar is equal to or less than the designated value, the electronic device 200 may perform operation 620 to measure the amount of change in blood sugar.

In operation 621, if the amount of blood sugar decreases at a rate exceeding the designated value, the electronic device 200 may determine, in operation 623, whether the blood sugar level is lower than a first threshold value. For example, the first threshold value may be configured to determine whether the electronic device 200 outputs a hypoglycemia prediction notification when hypoglycemia has not occurred in the user but the possibility of occurrence of hypoglycemia is high.

As a result of performing operation 623, if the blood sugar level is equal to or higher than the first threshold value, the electronic device 200 may perform operation 620 to measure the amount of change in blood sugar.

As the result of performing operation 623, if the blood sugar level is lower than the first threshold value, the electronic device 200 may determine movement information of the user in operation 630. For example, the movement information may include information indicating whether the user is exercising, sleeping, eating, or drinking.

In operation 640, the electronic device 200 may output a hypoglycemia prediction notification based on the movement information of the user.

According to various embodiments of the disclosure, if the blood sugar level of the user is lower than the first threshold value and the user is exercising, the electronic device 200 may determine that the user is temporarily in a hypoglycemia state due to the exercise. For example, the electronic device 200 may output, as the hypoglycemia prediction notification, information for encouraging the user to stop exercising and take a rest.

According to various embodiments of the disclosure, the electronic device 200 may include information of a proper exercise time in the hypoglycemia prediction notification. For example, because the blood sugar level is measured at a highest value when 1 hour has passed after a meal, the electronic device 200 may output the hypoglycemia prediction notification including information for recommending to exercise when the blood sugar level is the highest value after the meal.

According to various embodiments of the disclosure, the electronic device 200 may determine that the user's blood sugar level is lower than the first threshold value, and the user is in an inactive state. For example, the electronic device 200 may output, as the hypoglycemia prediction notification, a notification for recommending to increase blood sugar by taking glucose.

In operation 650, the electronic device 200 may determine whether the user's blood sugar level is measured to be less than a second threshold value. For example, the second threshold value is a value less than the first threshold value, and may be configured to a blood sugar level corresponding to occurrence of hypoglycemia.

As the result of performing operation 650, if the user's blood sugar level is lower than the second threshold value, the electronic device 200 may determine hypoglycemia has occurred in the user.

As the result of performing operation 650, if the user's blood sugar level is equal to or higher than the second threshold value, the electronic device 200 may perform operation 620 to measure the amount of change in blood sugar.

As the result of performing operation 650, if it is determined that the blood sugar level is less than the second threshold value within a designated time, the electronic device 200 may determine, in operation 660, that hypoglycemia has occurred in the user.

In operation 670, the electronic device 200 may transmit an emergency message (or an emergency notification and contact). For example, the emergency notification and contact may allow transmission of information informing occurrence of hypoglycemia of the user to a predesignated institution (e.g., a fire department or a hospital) or a predesignated contact.

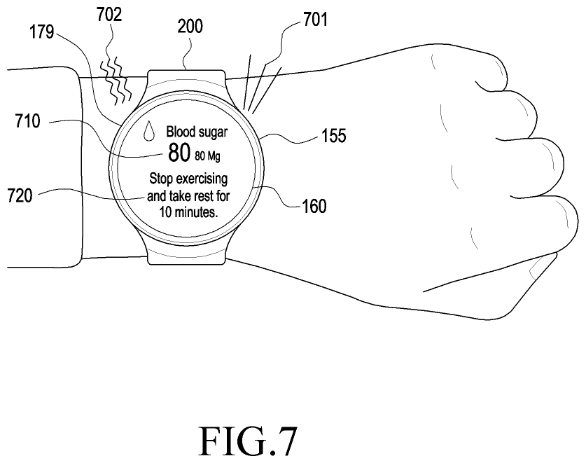

FIG. 7 is a diagram illustrating an example biometric information prediction notification output by the electronic device according to various embodiments of the disclosure.

Referring to FIG. 7, the electronic device 200 may include at least one among the display device 160, the sound output device 155, and the haptic module 179.

According to various embodiments of the disclosure, the electronic device 200 may measure an amount of user's blood sugar, and may output a hypoglycemia prediction notification based on the measured amount of blood sugar and movement information of the user. For example, the electronic device 200 may determine that the user is exercising, and a blood sugar level is lower than a threshold value (90 mg/dl) for outputting of the hypoglycemia prediction notification.

According to various embodiments of the disclosure, the electronic device 200 may determine that, even if the amount of blood sugar, which is measured for the user, does not indicate occurrence of hypoglycemia, because the user is exercising, the amount of blood sugar corresponds to a range (e.g., 70-80 mg/dl) lower than usual. For example, the electronic device 200 may control the output device 210 (e.g., the display device 160) to display, as the hypoglycemia prediction notification, information 710 of the current amount of blood sugar and a content 720 (e.g., “Stop exercising and take a rest for 10 minutes”.) informing the user to stop exercising.

According to various embodiments of the disclosure, the electronic device 200 may output the hypoglycemia prediction notification in various forms according to the movement information of the user. For example, when it is determined that the user is exercising and is thus unable to check the electronic device 200, the electronic device 200 may control the output device (e.g., the haptic module 179, the sound output device 155, or the output device 210 of FIG. 2) of the electronic device 200 to output non-visual data (e.g., sound data 701 or vibration data 702).

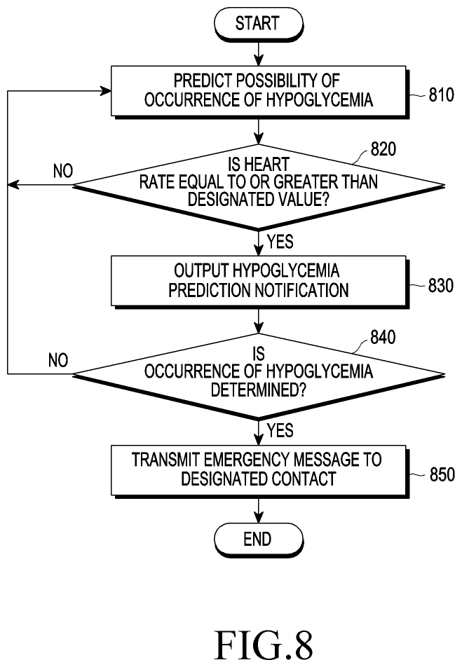

FIG. 8 is a flowchart illustrating an example operation of outputting a hypoglycemia prediction notification by the electronic device according to various embodiments of the disclosure.

Referring to FIG. 8, in operation 810, the electronic device 200 may predict a probability of occurrence of hypoglycemia for a user. For example, the electronic device 200 may monitor an amount of blood sugar, and if it is determined that the amount of a user's blood sugar decreases at a rate exceeding a designated value and a blood sugar level is higher than a value corresponding to hypoglycemia, the electronic device 200 may determine that hypoglycemia is likely to occur.

In operation 820, the electronic device 200 may measure a heart rate of the user and may determine whether the measured user's heart rate is equal to or greater than a designated value.

As the result of performing operation 820, if the user's heart rate is lower than the designated value, the electronic device 200 may predict the possibility of occurrence of hypoglycemia in operation 810.

As the result of performing operation 820, if the user's heart rate is equal to or greater than the designated value, the electronic device 200 may output a hypoglycemia prediction notification in operation 830.

According to various embodiments of the disclosure, the electronic device 200 may determine that the change in the heart rate is caused by the response of sympathetic nerves when the heart rate is equal to or greater than the designated value. The electronic device 200 may determine not only the change in the heart rate but also a change the amount of sweat, tremor, or breath.

In operation 840, the electronic device 200 may determine occurrence of hypoglycemia. For example, after the hypoglycemia prediction notification is output, if the amount of the user's blood sugar is measured to correspond to a value corresponding to hypoglycemia, the electronic device 200 may determine that hypoglycemia has occurred in the user.

In operation 850, the electronic device 200 may transmit an emergency message to a designated contact if an occurrence of hypoglycemia is determined in operation 840. For example, the designated contact may be the user's contact number preconfigured by the user, or may include a contact (e.g., 911) of a rescue agency to transmit an immediate rescue request.

According to various embodiments of the disclosure, the electronic device 200 may output the hypoglycemia prediction notification, and may monitor biometric information of the user to determine whether hypoglycemia occurs. Based on the determination, the electronic device 200 may not only output the notification but also perform control to make an emergency call to contacts (e.g., a rescue agency or a hospital) allowing urgent help for the user.

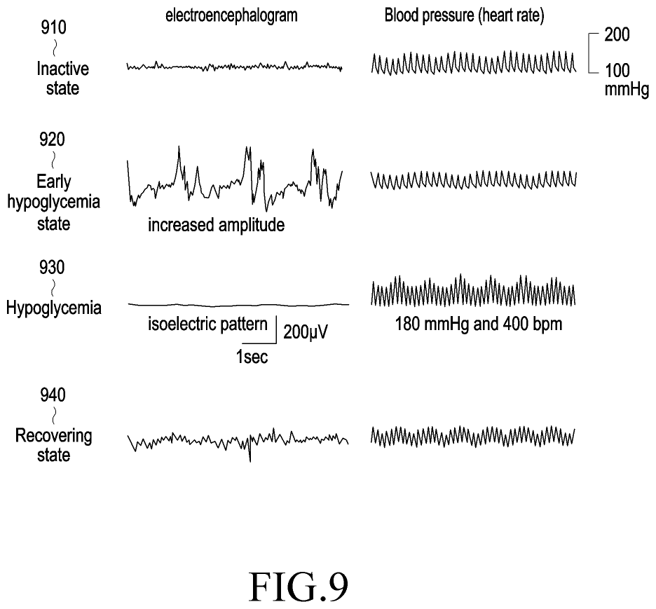

FIG. 9 is a graph illustrating examples of change in a biometric signal of a user when hypoglycemia occurs, according to various embodiments of the disclosure.

Referring to FIG. 9, the electronic device 200 may check changes in electroencephalogram, blood pressure, and heart rate according to a hypoglycemia occurrence state of a user.

According to various embodiments of the disclosure, if a user is in an inactive state 910, the electroencephalogram and blood pressure may be measured at a constant value without a change in the amplitude.

According to various embodiments of the disclosure, the electronic device 200 may check the amount of the user's blood sugar, and if the amount of the user's blood sugar has a value lower than a normal range but higher than a value for determination of hypoglycemia, the electronic device 200 may determine that the user is in an early hypoglycemia state 920. For example, in the early hypoglycemia state 920, the amplitude is increased compared to a graph of change in electroencephalogram for the inactive state 910, and a reduced amplitude may (e.g., 80 mmHg) be measured compared to a graph of change in blood pressure.

According to various embodiments of the disclosure, in the electronic device 200, a pattern (isoelectric pattern) with almost no change in electroencephalogram (e.g., 200 μV/sec) appears in a hypoglycemia state 930 due to the amount of the user's blood sugar, which is lower than a designated value, and the blood pressure and heart rate in the hypoglycemia state 930 may be measured to show increased amplitudes compared to an inactive (normal) state 910 and the early hypoglycemia state 920.

According to various embodiments of the disclosure, if hypoglycemia occurs in the user and then the blood sugar level is measured to be normal, the electronic device 200 may determine that the user is in a recovering state 940. In the recovering state 940, the amplitude of the electroencephalogram may be measured to be higher than that in the normal state 910, but may be measured to be lower than that in the early hypoglycemia state 920. The blood pressure in the recovering state 940 is similar to the pattern in the hypoglycemia state 930, but the amplitude may be measured to be low.

According to various embodiments of the disclosure, the electronic device 200 may determine a hypoglycemia occurrence state by means of a biometric signal measured for a user, based on graphs of changes in the biometric signal according to various hypoglycemia occurrence states.

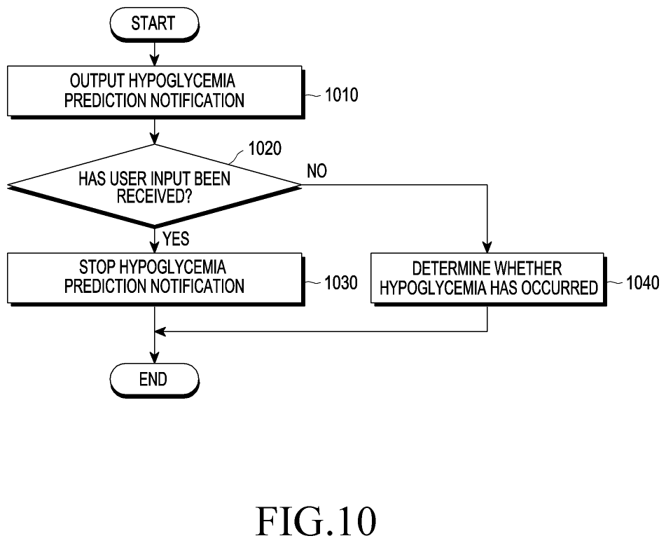

FIG. 10 is a flowchart illustrating an example operation of determining whether to output a hypoglycemia prediction notification, by the electronic device according to various embodiments of the disclosure.

Referring to FIG. 10, in operation 1010, the electronic device 200 may output a hypoglycemia prediction notification. The hypoglycemia prediction notification may be output if a blood sugar level of a user decreases at a rate equal to or higher than a designated value and has a value corresponding to a designated range.

In operation 1020, the electronic device 200 may determine whether a user input for the notification is received. For example, the electronic device 200 may configure, in advance, a type of the user input according to a situation of the user, which corresponds to movement information of the user.

According to various embodiments of the disclosure, the electronic device 200 may determine that the user is sleeping. For example, the electronic device 200 may determine whether the user is sleeping, by checking movement pattern information of the user, which is acquired by a sensor (e.g., an acceleration sensor) of the electronic device 200.

According to various embodiments of the disclosure, the electronic device 200 may determine that the user is sleeping, and may output the notification in various data forms in order to wake up the user. For example, the notification may include sound data or vibration data via a notification application. If it is determined that the user woke up within a designated time after the output of the notification, the electronic device 200 may determine that a designated user input has been received.

According to various embodiments of the disclosure, the electronic device 200 may determine that the user is in an inactive state. For example, the electronic device 200 may output a notification informing that, even if the user is in the inactive state, there may be a possibility of occurrence of hypoglycemia. For example, the notification may include a recommendation for immediate glucose intake.

According to various embodiments of the disclosure, the user may input a response to the notification, to the electronic device 200. For example, the user input may include a specific action (e.g., sitting) or gesture input, or voice data (e.g., “yes” or “alright”), and various forms of data which can be input via various input devices may be additionally possible.

As the result of performing operation 1020, if the user input for the notification is received, the electronic device 200 may stop outputting of the hypoglycemia prediction notification, in operation 1030. For example, the electronic device 200 may determine that the user has checked the notification and taken a self-measurement to increase blood sugar, and may measure an amount of blood sugar in the background.

As the result of performing operation 1020, if no user input predesignated for the notification is received, the electronic device 200 may determine whether hypoglycemia has occurred, in operation 1040. For example, the electronic device 200 may determine whether the user's blood sugar level has a value corresponding to hypoglycemia.

According to various embodiments of the disclosure, the electronic device 200 may output a hypoglycemia prediction notification, and may stop outputting the hypoglycemia prediction notification or may determine whether hypoglycemia has occurred in a user, according to whether a user input has been received in response to the notification.

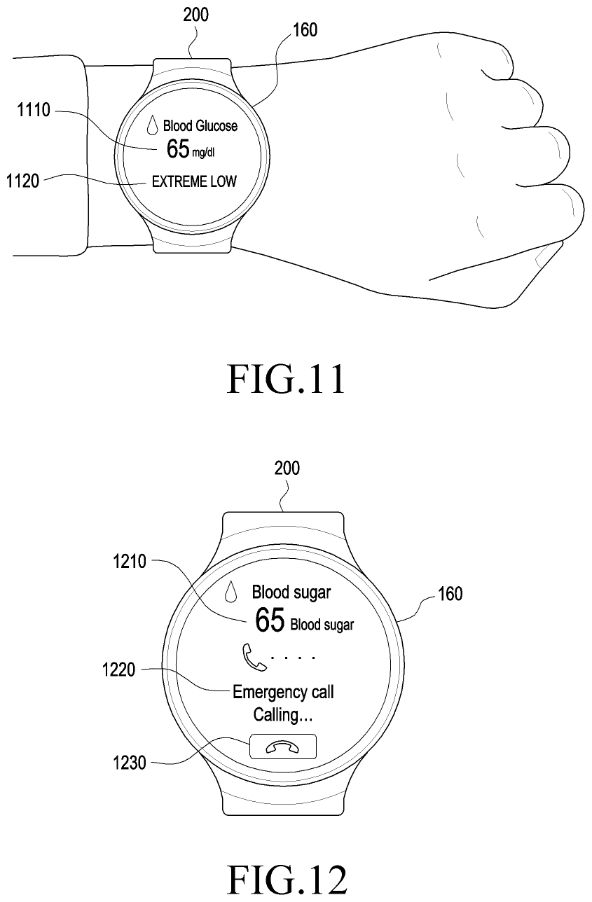

FIG. 11 is a diagram illustrating an example of a hypoglycemia prediction notification configuration in the electronic device according to various embodiments of the disclosure.

Referring to FIG. 11, the electronic device 200 may output a hypoglycemia occurrence notification via the display device 160.

According to various embodiments of the disclosure, the electronic device 200 may measure an amount of blood sugar of a user, and may determine whether the measured amount of blood sugar has a value corresponding to hypoglycemia. For example, if the amount of blood sugar is measured to be a value less than 70 mg/dl, it may be determined that hypoglycemia has occurred in the user.

According to various embodiments of the disclosure, the electronic device 200 may output various notifications informing occurrence of hypoglycemia in the user. For example, the notification may include information (e.g., “extremely low”) 1120 alerting that the user's blood sugar level (e.g., 65 mg/dl) 1110 or the amount of blood sugar is extremely low.

According to various embodiments of the disclosure, the electronic device 200 may determine whether a user input is made within a designated time after the hypoglycemia occurrence notification is output. For example, the user input may include various types of data indicating whether the user has checked the notification.

FIG. 12 is a diagram illustrating an example of a hypoglycemia occurrence notification configuration in the electronic device according to various embodiments of the disclosure.

Referring to FIG. 12, if it is determined that hypoglycemia has occurred in a user, the electronic device 200 may transmit a call to an emergency contact.

According to various embodiments of the disclosure, the electronic device 200 may determine that hypoglycemia has occurred in the user when a blood sugar level 1210 of the user is measured to be lower than a value (e.g., 70 mg/dl) corresponding to hypoglycemia. For example, the electronic device 200 may display, via the display device 160, a current blood sugar level 1210, an emergency call origination notification 1220, and a button 1230 that allows termination of a call.

According to various embodiments of the disclosure, the electronic device 200 may transmit an emergency call (e.g., 911) or may transmit a message including information related to occurrence of hypoglycemia in the user to a designated contact, according to whether movement information of a user or a user input for a previous hypoglycemia prediction notification has been received.

For example, if movement information of the user cannot be checked, there is no movement change, or no record for reception of a user input for the hypoglycemia prediction notification within a designated time, the electronic device 200 may determine that the user is unable to perform self-measurement for hypoglycemia and may transmit an emergency call.

According to various embodiments of the disclosure, if the user has recovered from hypoglycemia or has failed to check the emergency call origination notification 1220, the user may select the button 1230 to stop the emergency call. For example, when the button 1230 is selected, the electronic device 200 may stop transmitting the emergency call, and periodically measure the user's blood sugar to determine whether hypoglycemia occurs in the user.

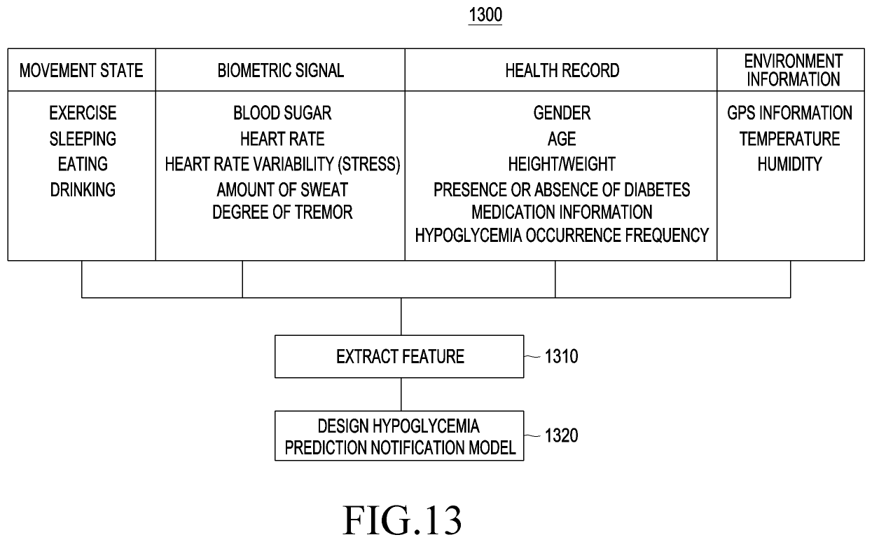

FIG. 13 is a diagram illustrating an example data configuration for designing a hypoglycemia prediction notification model by the electronic device according to various embodiments of the disclosure.

Referring to FIG. 13, the electronic device 200 may check data 1300 related to a user. For example, the data 1300 may include the user's movement state, biometric signal, health record, or environment information.

According to various embodiments of the disclosure, the movement state may include information indicating a current activity state of the user, which indicates whether the user is exercising, sleeping, eating, drinking, or the like. The biometric signal may include biometric information of the user, which is measured via a sensor module 176 of the electronic device 200, and may include information indicating blood sugar, heart rate, heart rate variability (stress), amount of sweat, degree of tremor, or the like. The health record may include information related to the user's health, and may include information, such as gender, age, height/weight, the presence or absence of diabetes, medication information, or a frequency of hypoglycemia. The environment information may include information of the user's surrounding environment and may include GPS information, temperature information, or humidity information.

According to various embodiments of the disclosure, in operation 1310, the electronic device 200 may extract various features related to hypoglycemia based on the data 1300. For example, the various features related to hypoglycemia may include a blood sugar level for occurrence of hypoglycemia, a rate value of decrease in blood sugar, hypoglycemia occurrence context information, or the like.

According to various embodiments of the disclosure, the electronic device 200 may determine movement information of the user and a blood sugar level measured for the user, and if the movement information and blood sugar level correspond to the extracted feature, the electronic device may determine to output the hypoglycemia prediction notification.

According to various embodiments of the disclosure, in operation 1320, the electronic device 200 may design a hypoglycemia prediction notification model. For example, the hypoglycemia prediction notification model may include various configurations related to output of the hypoglycemia prediction notification, such as a notification output period, an output time, a type of data included in the notification, a blood sugar measurement period, an output method, etc., for each of the extracted various features.

According to various embodiments of the disclosure, the electronic device 200 may extract various features for determining occurrence of hypoglycemia, and may perform modeling for extracting a hypoglycemia prediction notification therefor, based on the data 1300 for a user.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

While the disclosure has been illustrated and described with reference to various example embodiments, it will be understood that the various example embodiments are intended to be illustrative, not limiting. Accordingly, one of ordinary skill in the art will understand that various changes in form and detail may be made without departing from the scope of the disclosure, including the appended claims and their equivalents.

What is claimed is:

1. An electronic device comprising:
a sensor module;
an output device comprising output circuitry;
a processor operatively connected to the sensor module and the output device; and
a memory operatively connected to the processor,
wherein the memory is configured to store instructions which, when executed, cause the processor to control the electronic device to:
identify a blood sugar level measured at every first period via the sensor module;
output a hypoglycemia prediction notification via the output device based on an amount of change in decrease of the blood sugar level exceeding a designated first value; and
measure a blood sugar level via the sensor module at every second period, the second period being shorter than the first period, based on a blood sugar level identified after the notification is output being equal to or less than a designated second value.

2. The electronic device of claim 1, wherein the instructions, when executed, cause the processor to control the electronic device to output, via the output device, a first notification informing the occurrence of hypoglycemia based on occurrence of hypoglycemia being determined based on the blood sugar level measured at every second period.

3. The electronic device of claim 2, wherein the instructions, when executed, cause the processor to control the electronic device to output the first notification via the output device at every second period.

4. The electronic device of claim 2, wherein the instructions, when executed, cause the processor to control the electronic device to output the first notification via the output device at every third period corresponding to the amount of change in decrease of the blood sugar level.

5. The electronic device of claim 1, further comprising a communication module comprising communication circuitry, wherein the instructions, when executed, cause the processor to control the electronic device to control the communication module to transmit an emergency call based on a user input for the first notification not being received within a predesignated time.

6. The electronic device of claim 1, wherein the output device further comprises a haptic module comprising haptic circuitry or a sound output device comprising sound output circuitry, and a data type included in the hypoglycemia prediction notification comprises at least one of vibration data output via the haptic module, sound data output via the sound output device, text output via the display device, or image data output via the display device.

7. The electronic device of claim 6, wherein the instructions, when executed, cause the processor to control the electronic device to:
determine, via the sensor module, that a user is in a sleeping state; and
in the sleeping state in which the user is sleeping, output the voice data or the vibration data, which is configured to induce the user to wake up, via the output device based on a rate of decrease in the blood sugar level measured at every first period exceeding a designated value.

8. The electronic device of claim 7, wherein the instructions, when executed, cause the processor to control the electronic device to output the hypoglycemia prediction notification comprising a content that is configured to induce glucose intake, via the output device based on determining, via the sensor module, that the user is in a wake-up state.

9. The electronic device of claim 1, wherein the instructions, when executed, cause the processor to control the electronic device to output the hypoglycemia prediction notification via the output device at each configured period based on a predesignated user input not being received within a designated time after the hypoglycemia prediction notification is output.

10. The electronic device of claim 1, wherein the instructions, when executed, cause the processor to control the electronic device to:
measure a biometric signal via the sensor module; and
determine whether hypoglycemia occurs based on at least a part of a pattern of the measured biometric signal.

11. A method for providing a biometric information notification by an electronic device, the method comprising:
identifying a blood sugar level measured at every first period;
outputting a hypoglycemia prediction notification based on an amount of change in decrease of the blood sugar level exceeding a designated first value; and
measuring a blood sugar level at every second period, the second period being shorter than the first period, based on a blood sugar level identified after the notification is output being equal to or lower than a designated second value.

12. The method of claim 11, further comprising: outputting a first notification informing the occurrence of hypoglycemia based on occurrence of hypoglycemia being determined based on the blood sugar level measured at every second period.

13. The method of claim 12, further comprising: outputting the first notification at every second period.

14. The method of claim 12, further comprising: outputting the first notification via the output device at every third period corresponding to an amount of change in decrease of the blood sugar level.

15. The method of claim 11, further comprising: transmitting an emergency call based on a user input for the first notification not being received within a predesignated time.

16. The method of claim 11, wherein a data type included in the hypoglycemia prediction notification comprises at least one of vibration data output via a haptic module, sound data output via a sound output device, text output via a display device, or image data output via the display device.

17. The method of claim 16, further comprising:
determining that the user is in a sleeping state; and
in the sleeping state in which the user is sleeping, outputting the voice data or the vibration data configured to induce the user to wake up based on a rate of decrease in the blood sugar level measured at every first period exceeding a designated value.

18. The method of claim 17, further comprising: outputting the hypoglycemia prediction notification comprising a content configured to induce glucose intake based on determining that the user is in a wake-up state.

19. The method of claim 11, further comprising: outputting the hypoglycemia prediction notification at each configured period based on a predesignated user input not being received within a designated time after the hypoglycemia prediction notification is output.

20. The method of claim 11, further comprising:
measuring a biometric signal; and
determining whether hypoglycemia occurs based on at least a part of a pattern of the measured biometric signal.
